(19) United States
(12) Patent Application Publication
(10) Pub. No.: US 2026/0151802 A1
(43) Pub. Date: Jun. 4, 2026

(54) Laparoscopic lens cap with ultrasonic cleaning system

(71) Applicants: Anthony Wong, Urbana, IL (US); Sanskruthi Priya Guduri, Palatine, IL (US); Asaiah Rock, Lake Zurich, IL (US); Debora Nya, Acworth, GA (US); Annie Tigranyan, Herndon, VA (US); Prakhar Gupta, Champaign, IL (US); Urvshi Thapar, Champaign, IL (US)

(72) Inventors: Anthony Wong, Urbana, IL (US); Sanskruthi Priya Guduri, Palatine, IL (US); Asaiah Rock, Lake Zurich, IL (US); Debora Nya, Acworth, GA (US); Annie Tigranyan, Herndon, VA (US); Prakhar Gupta, Champaign, IL (US); Urvshi Thapar, Champaign, IL (US)

(21) Appl. No.: 18/964,666

(22) Filed: Dec. 1, 2024

(51) Int. Cl. B08B 7/02; A61B 1/00; A61B 1/313; B06B 1/06

(52) U.S. Cl. CPC B08B 7/028; A61B 1/00096; A61B 1/3132; B06B 1/0655; B06B 2201/76

(57) ABSTRACT

A system for cleaning a laparoscopic lens cap and methods directed thereto is provided. The system includes a lens cap including a hydrophobic-coated optic window, a piezoelectric transducer, a transducer control system, a piezoelectric driver, a power supply like a wall adapter or battery, and a power switch. In operation, the user of a laparoscope detects an obstruction on the optic window and activates the power switch. The power source provides electrical power to the transducer control system and driver, which further transmits the electrical power to the piezoelectric transducer. The piezoelectric transducer converts this electrical power via a voltage signal into mechanical displacement, which, due to the ultrasonic frequency of the waveform, produces acoustic waves. These acoustic waves serve as mechanical forces that are able to forcibly remove any obstruction that is detected on the lens cap with the additional aid of vibrational effects on the lens cap.

### BACKGROUND OF THE INVENTION

The present invention generally relates to systems for cleaning laparoscopes and methods for using such systems. More particularly, the present invention relates to systems that ultrasonically clean a laparoscopic lens cap and methods for using such systems.

Maintaining the cleanliness of laparoscopes is important to medical professionals. For example, laparoscopes provide surgeons with a clear view inside a patient's body while the surgeons are performing a medical procedure. During surgery, bodily fluids or tissue products may obscure the surgeon's view by splashing on or otherwise obscuring or adhering to the lens of the laparoscope. In addition, water condensation or fog can also obscure the surgeon's view. These obstructions lead to poor visibility and require the surgeons to perform surgery in a stressful situation, which increases the chances of surgical injuries. Indeed, poor visualization is the cause of 1 in 5 surgical injuries. Most surgeries require removal of the laparoscope for cleaning, which can take up to 82 seconds of surgery time and may cause the patient to lose up to 200 milliliters of blood.

Several prior art systems exist to clean laparoscopes. One example of a prior art system is Baril et al., U.S. Pat. No. 11,850,106 which discloses a cleaning cap for surgical devices. In this prior art system, an obstructed surgical instrument is removed from the patient's body and the cleaning cap, which includes wipers, is attached to the obstructed end of the surgical instrument. The wipers wipe away the obstruction and the surgical instrument can be reinserted into the patient.

The prior art system of Fiset, U.S. Pat. No. 10,939,812 also discloses an external cleaning device system to clean a surgical instrument which includes a sponge and heating element. The surgical scope is heated by the heating element and wiped with the sponge to remove any debris.

Another example includes the wiper device disclosed in Sheffield et al., U.S. Patent App. Pub. US 2023/0240522A1. In the system of Sheffield et al., an optical instrument is fitted with a sheath including a folded wiper blade and an actuator. The actuator maintains tension on the wiper blade, keeping the wiper blade in its folded state. When the lens of the optical instrument is obstructed, the actuator releases the tension on the wiper blade, enabling the wiper blade mechanism to spring across the lens and push off the obstruction.

Another prior art system disclosed in Drach et al., U.S. Pat. No. 9,211,059 prevents water condensation from obscuring the lens of surgical scopes. In this system, the surgical scope is fitted with a sheath that flows CO₂ gas and prevents the lens from fogging up and obscuring visibility.

In another example, the prior art system of Hsu et al., U.S. Pat. No. 8,979,738 includes a sheath for the objective lens of an endoscope. In this system, a user observes an obstruction on the lens of the endoscope and clicks a button, which releases a microfilm over the objective lens of the laparoscope. In one embodiment of Hsu et al., the used microfilm is removed from the system by wrapping the used microfilm around a spool.

Another prior art system includes an ultrasonic lens cleaning technology for applications such as reverse driving cameras. For example, Magee et al., U.S. Pat. No. 11,237,387 describes a lens cleaning system including a transducer that vibrates a lens to blast off the debris.

### BRIEF SUMMARY OF THE INVENTION

One or more of the embodiments of the present invention provide systems and methods for cleaning a laparoscopic lens. One system for cleaning a laparoscopic lens includes a laparoscope and a lens cap. The lens cap comprises a piezoelectric transducer, a transducer control system, a piezoelectric driver, a power source, and a switch.

In the system for cleaning a laparoscopic lens, the lens cap is mechanically connected to the objective lens of the laparoscope. Additionally, the piezoelectric transducer, the transducer control system, the power source, the piezoelectric driver and switch are in electrical connection with each other.

In operation, the medical professional performing a laparoscopy observes debris (e.g., blood, tissue, or other bodily fluids) on the lens cap affixed to the laparoscope. The user activates the switch to an “on” position. As a result, the power source begins to provide power to the system. Specifically, the transducer control system and the piezoelectric driver receive electrical power. The piezoelectric driver provides electrical power to the piezoelectric transducer at a specified ultrasonic resonant frequency. Upon receipt of electrical power, the piezoelectric transducer converts this electrical power to mechanical displacement, which further produces an acoustic wave with a frequency equivalent to that of the waveform of the driving signal sent from the piezoelectric driver. The acoustic wave moves forward (e.g., away from the piezoelectric transducer) until it strikes the debris. As the piezoelectric transducer vibrates ultrasonically, the debris is displaced from the lens cap. Alternatively, the debris no longer adheres to the lens cover.

### BRIEF DESCRIPTION OF THE DRAWINGS

FIG. 1 illustrates a cross-section view of a laparoscopic lens cap with an ultrasonic cleaning system.

FIG. 2 illustrates a flow chart of a method for displacing an obstruction from a laparoscopic lens cap.

FIG. 3 illustrates a block diagram of a system for ultrasonically cleaning a laparoscopic lens cap according to an embodiment of the present invention.

FIG. 4 illustrates a side view of an ultrasonic cleaning system for a laparoscopic lens.

FIG. 5 illustrates a cross-section view of an alternative embodiment system for ultrasonically cleaning a laparoscopic lens cap.

### DETAILED DESCRIPTION OF THE INVENTION

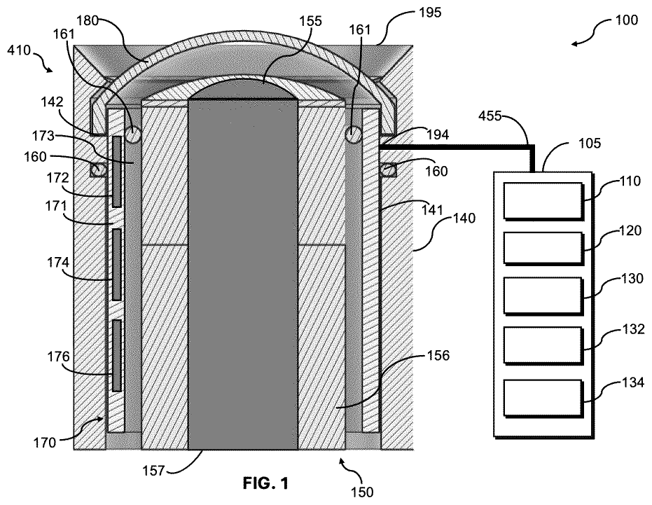

FIG. 1 illustrates a cross-section view of a laparoscopic lens cap with an ultrasonic cleaning system 100 according to an embodiment of the present invention. The laparoscopic lens cap with an ultrasonic cleaning system 100 includes a power system 105, a laparoscopic lens cap 410, and a laparoscope 150. The laparoscopic lens cap 410 includes an exterior housing 140, a funnel guard 195, a hydrophobic-coated optic window 180, a piezoelectric transducer 170, an optic window recess 142, a power wire 455, a cap-sealing O-ring 160, an interior housing O-ring 161 and an interior housing wall 141. The piezoelectric transducer 170 includes a lower electrode 172, a piezoelectric material 174, an upper electrode 176, a cylindrical transducer housing 171, an interior transducer wire aperture 194 and a piezoelectric transducer contact location 173. The laparoscope 150 includes a laparoscopic outer wall 156, a laparoscopic interior 157, and an objective lens 155. The power system 105 includes a power source 110, a power switch 120, a transducer control system 130, a printed circuit board (PCB) 132, and a piezo driver 134.

In the laparoscopic lens cap with an ultrasonic cleaning system 100, the power source 110, the power switch 120, the transducer control system 130, the PCB 132, the piezo driver 134, and the piezoelectric transducer 170 are in an electrical connection with each other. Specifically, the cylindrical transducer housing 571 further comprises an interior transducer wire aperture 194. The piezoelectric transducer 170 is electrically coupled to the power system 105 via power wire 455. The piezoelectric transducer 170 is mechanically connected to the power wire 455 through the interior transducer wire aperture 194, which passes through the cylindrical transducer housing 171. The piezoelectric transducer 170 includes a lower electrode 172, a piezoelectric material 174, and an upper electrode 176; the lower electrode 172, the piezoelectric material 174, and the upper electrode 176 are positioned inside the cylindrical transducer housing 171. The cylindrical transducer housing 171 is mechanically affixed to the interior housing wall 141 at the piezoelectric transducer contact location 173. The interior housing O-ring 161 is positioned between the cylindrical transducer housing 171 and the exterior housing 140, causing the cylindrical transducer housing 171 to be mechanically affixed to the interior housing wall 141. The cylindrical transducer housing 171 is also frictionally affixed to the laparoscopic outer wall 156. The cap-sealing O-ring 160 is positioned between the cylindrical transducer housing 171 and the laparoscopic outer wall 156, forming a watertight seal between the cylindrical transducer housing 171 and the laparoscopic outer wall 156. The exterior housing 140 is in mechanical connection with the funnel guard 195 and the hydrophobic-coated optic window 180. The hydrophobic-coated optic window 180 is held in place by the exterior housing 140 and the interior housing wall 141 such that the hydrophobic-coated optic window 180 is positioned in the optic window recess 142. The exterior housing 140 is also in mechanical connection with the laparoscope 150, specifically at the laparoscopic outer wall 156. The laparoscopic outer wall 156 is mechanically connected to the laparoscopic interior 157. The laparoscopic interior 157 is mechanically coupled with the objective lens 155.

In operation, the laparoscope 150 and the laparoscopic lens cap 410 are frictionally affixed, causing the hydrophobic-coated optical window 180 to be radially offset from the objective lens 155 of the laparoscope 150. Next, the laparoscope 150 is inserted into a patient's body by a user such as a medical professional. When the hydrophobic-coated optic window 180 is obscured by an obstruction or debris (e.g., blood, tissue, or other bodily fluids) on the surface of the hydrophobic-coated optical window 180, the user activates the power switch 120 into an “on” position. Once the power switch is activated, the power source 110 begins providing electrical power to the PCB 132 and the transducer control system 130. In turn, the transducer control system 130 provides electrical power to the piezo driver 134. The piezo driver 134 further provides electrical power to the piezoelectric transducer 170.

In further operation, the piezoelectric transducer 170 includes a lower electrode 176 and an upper electrode 172 with a piezoelectric material 174 (e.g., barium titanate, lead zirconate titanate (PZT), lead titanate, Rochelle salt) situated between the two electrodes. Once powered, the piezoelectric material vibrates at a resonant frequency provided by the piezoelectric driver for a specified voltage, powered by the power source, causing the piezoelectric transducer 170 to vibrate at an ultrasonic frequency (e.g., 20 kHz-400 kHz). As the piezoelectric transducer 170 ultrasonically vibrates, the debris/obstruction on the hydrophobic-coated optic window 180 is displaced from the surface of the hydrophobic-coated optic window 180. Alternatively, the debris/obstruction no longer adheres to the lens/is physically or mechanically separated from the lens. In embodiments that include a funnel guard 195, the guard guides the debris away from the hydrophobic-coated optic window 180 and back into the body.

In one embodiment, the cylindrical transducer housing 171 is frictionally affixed to the laparoscopic outer wall 156 through mechanical retention. It is further sealed by applying pressure to the cap-sealing O-ring 160. The cap-sealing O-ring 160 is positioned between the cylindrical transducer housing 171 and the laparoscopic outer wall 156, forming a watertight seal between the cylindrical transducer housing 171 and the laparoscopic outer wall 156. When the cylindrical transducer housing 171 is affixed to the laparoscopic outer wall 156, the hydrophobic-coated optical window 180 is radially offset from the objective lens 155 of the laparoscope 150 such that the objective lens 155 includes a field of view through the hydrophobic-coated optic window 180.

In the preferred embodiment, the hydrophobic-coated optic window 180 is coated in a hydrophobic coating which reduces the adhesion of debris/obstruction, defogs the lens, and is both hydrophobic and oleophobic such as Teflon.

In the preferred embodiment, the transducer control system 130 is an integrated circuit.

In the preferred embodiment, the cylindrical transducer housing 171 is mechanically affixed to the interior housing wall 141 at the piezoelectric transducer contact location 173. The hydrophobic-coated optic window is affixed between the exterior housing 140 and the interior housing wall 141, causing the hydrophobic-coated optic window 180 to be positioned within the optic window recess 142.

In the preferred embodiment, the piezoelectric transducer 170 is a ceramic piezoelectric transducer.

In the preferred embodiment, the piezoelectric transducer 170 is coated in a Bakelite resin for sterilization.

In the preferred embodiment, the piezoelectric transducer 170 is a hollow cylinder that is in mechanical connection with the objective lens 155 of the laparoscope 150.

In the preferred embodiment, the exterior housing 140 is composed of medical-grade silicone. The hydrophobic-coated optic window 180 is mechanically fitted into the exterior housing 140 such that the housing mechanically couples the hydrophobic-coated optic window 180 in place. The cap sealing O-ring 160 further mechanically couples the hydrophobic-coated optic window 180 in place.

In the alternative embodiment, the exterior housing 140 may be composed of medical-grade plastic such as polyethylene, polypropylene or polycarbonate, or surgical stainless steel.

In the preferred embodiment, the piezo driver 134 is the Texas Instruments DRV2901 PWM-Input Piezoelectric Transducer Driver.

In the preferred embodiment, the power source 110 is an external electrical power source such as a wall outlet that provides electrical power.

In an alternative embodiment, the laparoscopic lens cap 410 is single-use.

In an alternative embodiment, the power supply for the power system 105 is supplied by the power source of the laparoscope 150.

In an alternative embodiment, instead of using power source 110, the system for ultrasonically cleaning laparoscopic lens cap 300 includes a battery which provides sufficient power for the device. Once received by the piezo driver 134, that power is then distributed to the piezoelectric transducer 170.

In one embodiment, the piezoelectric transducer 170 is a piezoelectric actuator. In an alternative embodiment, the piezoelectric transducer 170 is a piezoelectric actuator and piezoelectric sensor.

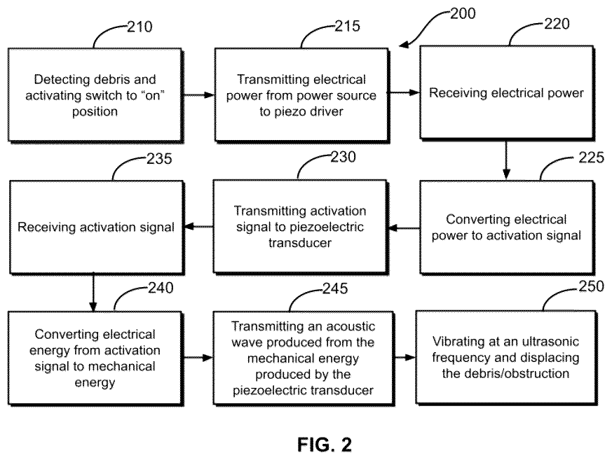

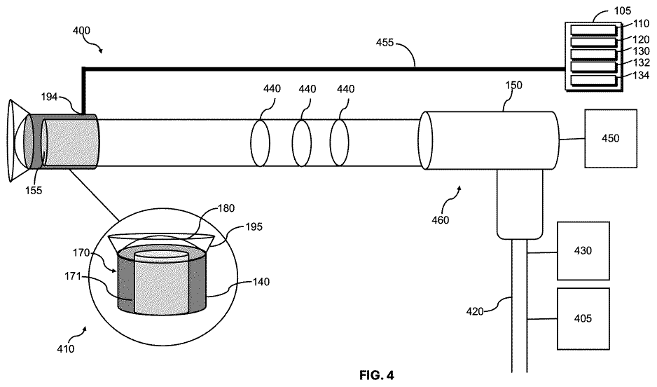

FIG. 2 illustrates a method for ultrasonically cleaning a laparoscopic lens cap. First, at step 210, a user detects debris (e.g., blood, fat, tissue, or other bodily fluids) or an obstruction obscuring the hydrophobic-coated optic window 180 (as described previously in FIG. 1) after viewing the camera 450 (as shown below in FIG. 4) of the laparoscope 150. When the user detects this debris or obstruction, the user turns the power switch 120 (as previously described in FIG. 1) to an “on” position. For example, the user can press a button or flip a toggle switch to activate an “on” position.

Next, at step 215, the battery 110 (as previously described in FIG. 1) will begin to provide electric power once the power switch 120 (as described above in FIG. 1) is in the “on” position, causing the circuit to be closed. The printed circuit board (PCB) 132 and the transducer control system 130 (as previously described in FIG. 1) receives this electrical power from the battery 110.

Next, at step 220, the piezo driver 134 receives electrical power from the transducer control system 130. At step 225, the piezo driver 134 converts this electrical power from the power source 110 into an electrical signal instructing the piezoelectric transducer 170 (as described previously in FIG. 1) to activate. This electrical signal is referred to as the “activation signal.” At step 230, the transducer control system 130 transmits this activation signal to the piezoelectric transducer 170.

Next, at step 235, the piezoelectric transducer 170 receives the activation signal. Upon receipt of the activation signal, a voltage potential (equivalent to the voltage potential sent from the piezo driver 134) is applied to the piezoelectric material 174 (as described above in FIG. 1) of the piezoelectric transducer 170. Consequently, at step 240, the piezoelectric material 174 rapidly compresses and expands, producing a vibration. This rapid compression and expansion produces acoustic waves which are projected outward from the piezoelectric transducer 170.

Next, at step 245, the acoustic wave is transmitted from the piezoelectric transducer 170. The acoustic wave moves forward until it makes contact with the debris or obstruction blocking the optic window 180. At step 250, the piezoelectric transducer 170 vibrates at an ultrasonic frequency. The resulting ultrasonic vibrations created by the piezoelectric material 174 displaces the debris or obstruction from the optic window 180.

In an alternative embodiment, the debris or obstruction observed on the lens cap by the user in step 210 is detected automatically. In this example, the piezoelectric transducer 170 includes both a piezoelectric actuator and a piezoelectric sensor. When the debris/obstruction makes contact with the hydrophobic-coated optic window 180, the piezoelectric sensor detects the debris/obstruction by sensing the mechanical vibrations of the contact through the generation of voltage via the piezoelectric effect. The piezoelectric material 174 converts the resulting mechanical energy (e.g., stress energy due to the displacement caused from the impact of the debris/obstruction on the hydrophobic-coated optic window 180) to electric voltage.

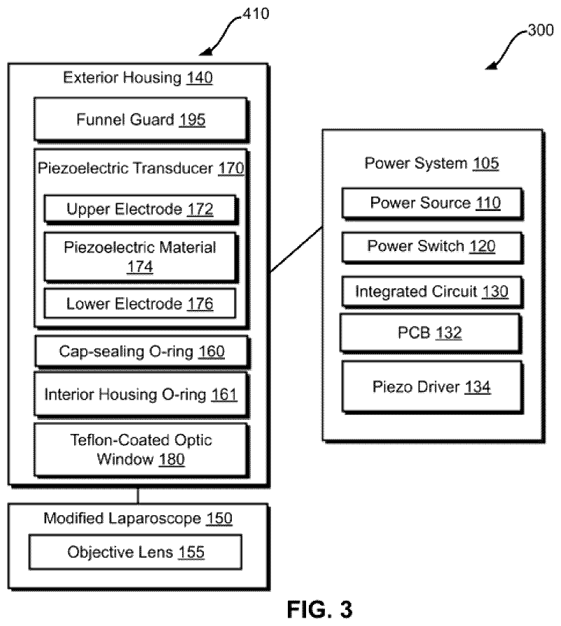

FIG. 3 illustrates a block diagram of a system for ultrasonically cleaning a laparoscopic lens cap 300 according to an embodiment of the present invention. The system for ultrasonically cleaning a laparoscopic lens cap 300 includes a power system 105, a laparoscopic lens cap 410, a laparoscope 150. The power system 105 includes a power source 110, a power switch 120, a transducer control system 130, a printed circuit board (PCB) 132, and a piezo driver 134. The laparoscopic lens cap 410 includes an exterior housing 140, a funnel guard 195, a hydrophobic-coated optic window 180, a piezoelectric transducer 170, the cap-sealing O-ring 160, and the interior housing O-ring 161. The piezoelectric transducer 170 includes an upper electrode 172, a piezoelectric material 174, a lower electrode 176, and a cylindrical transducer housing 171. The laparoscope 150 also includes an objective lens 155.

In the system for ultrasonically cleaning a laparoscopic lens cap 300, the power system 105 is in a wired connection with the piezoelectric transducer 170. Specifically, the power source 110, the power switch 120, the transducer control system 130, the PCB 132, the piezo driver 134 and the piezoelectric transducer 170 are in electrical connection with each other. The exterior housing 140 houses the piezoelectric transducer 170 as well as other components including the cap-sealing O-ring 160, the interior housing O-ring 161 and the hydrophobic-coated optic window 180. The exterior housing 140 is further mechanical connection with the funnel guard 195.

In addition, the exterior housing 140 is in further mechanical connection with the laparoscope 150, specifically the objective lens 155. Specifically, the exterior housing 140 is frictionally affixed to the laparoscope 150 such that the objective lens 155 of the laparoscope 150 includes a field of view through the hydrophobic-coated optic window 180.

In operation, the laparoscopic lens cap 410 is frictionally affixed to the objective lens 155 of the laparoscope 150. Once the laparoscopic lens cap 410 is coupled with the laparoscope 150, the laparoscope 150 is inserted into the patient's body. When a medical professional performing the laparoscopy views debris through the objective lens 155 of the laparoscope 150, the user activates the power switch 120.

Once the power switch 120 is activated, the power source 110 (e.g., a 120V US wall power source) activates and provides power to the transducer control system 130. Once power is received by the transducer control system 130, the electrical power is then distributed to the piezoelectric transducer 170. The piezoelectric transducer 170 includes a lower electrode 176 and an upper electrode 172 with a piezoelectric material 174 (e.g., barium titanate, lead zirconate titanate (PZT), lead titanate, Rochelle salt) situated between the two electrodes. Once powered, the piezoelectric material 176 resonates at an ultrasonic frequency equivalent to the voltage potential of the battery, causing the piezoelectric transducer to vibrate at an ultrasonic frequency (e.g., 20 kHz-400 kHz). As the piezoelectric transducer 170 ultrasonically vibrates, the debris/obstruction on the hydrophobic-coated optic window 180 is removed. Alternatively, the debris/obstruction no longer adheres to the lens/is physically or mechanically separated from the lens. In embodiments that include a funnel guard 195, the guard guides the debris away from the hydrophobic-coated optic window 180 and back into the body.

In an alternative embodiment, there is no funnel guard 195. Instead, debris/obstruction is cleared from the optic window solely through ultrasonic vibrations or through other mechanical means.

In an alternative embodiment, instead of using power source 110, the system for ultrasonically cleaning laparoscopic lens cap 300 includes an external electrical power source such as a wall outlet that provides electrical power. Once received by the piezo driver 134, that power is then distributed to the piezoelectric transducer 170.

In an alternative embodiment, the system for ultrasonically cleaning laparoscopic lens cap 300 is automatic, rather than manual. In this embodiment, the hydrophobic-coated optic window 180 is mechanically connected to the piezoelectric transducer 170, which is both a piezoelectric actuator and a piezoelectric sensor. Once debris or an obstruction sprays or adheres onto the hydrophobic-coated optic window 180, the piezoelectric sensor compresses due to the pressure from the contact of this debris/obstruction and generates a voltage proportional to the generated pressure. The transducer control system 130 receives this generated voltage and afterward sends a signal to the piezo driver 134 to activate. In response, the piezo driver 134 transmits an activation signal to the piezoelectric actuator, which converts the resulting electrical energy from the voltage generated back into mechanical energy. As described above, the piezoelectric material 174 then produces an acoustic wave that moves outward towards the debris/obstruction and makes contact with it. The piezoelectric material 174 of the piezoelectric transducer 120 will continue to vibrate ultrasonically for a designated length of time, and the debris/obstruction on the hydrophobic-coated optic window 180 is displaced or stops adhering to the surface of the hydrophobic-coated optic window 180.

In an alternative embodiment, the system for ultrasonically cleaning laparoscopic lens cap 300 includes a piezoelectric controller to control the frequency of the ultrasonic vibrations of the piezoelectric transducer 170.

FIG. 4 illustrates a side view of an ultrasonic combined cleaning system for a laparoscopic lens cap 400. The ultrasonic combined cleaning system for a laparoscopic lens cap 400 includes a power system 105, a laparoscopic lens cap 410, a laparoscopic assembly 460, and a power wire 455. The laparoscopic lens cap 410 includes an exterior housing 140, a funnel guard 195, a hydrophobic-coated optic window 180, and a piezoelectric transducer 170. The piezoelectric transducer 170 includes a cylindrical transducer housing 171 and an interior transducer wire aperture 194. The power system 105 includes a power source 110, a power switch 120, a transducer control system 130, a printed circuit board (PCB) 132, and a piezo driver 134. The laparoscopic system 105 also includes a laparoscope 150, an external power source 405 for the laparoscope 150, relay lenses 440, a fiber optic cable 420, a light source 430, a camera 450, and an objective lens 155.

The laparoscope 150 is mechanically connected to the system for ultrasonically cleaning a laparoscopic lens cap 400, specifically by threading the exterior housing 140 onto the tip of the laparoscope 150 (as described above in FIG. 3). The exterior housing 140 is further mechanically connected to the piezoelectric transducer 170 and the hydrophobic-coated optic window 180. The piezoelectric transducer 170 is in a wired connection with the power system 105 via power wire 455. The power wire 455 passes through the cylindrical transducer housing 571 via the interior transducer wire aperture 194. The power wire 455 further mechanically connects to the power system 105. The hydrophobic-coated optic window 180 is mechanically connected to the funnel guard 195. The laparoscope 150 is mechanically connected to an external power source 405, relay lenses 440, a fiber optic cable 420, a light source 430, and a camera 450. The power switch 120, the power source 110, the transducer control system 130, the PCB 132, and the piezo driver 134 are in electrical connection with each other.

In operation, the laparoscope 150 is frictionally affixed to the laparoscopic lens cap 410. The laparoscope 150 is powered by an external power source 405 (e.g., a battery or wall outlet) which provides electrical power to the laparoscope 150. The laparoscope 150 includes multiple lenses spaced throughout the laparoscope stem. The laparoscope is further attached to a light source 430 using the fiber optic cable 420 and the laparoscope 150 is inserted into a patient's body. When the light source 430 is powered on, the fiber optic cable 420 transmits light into the body through the multiple relay lenses 440 spaced along the laparoscope stem, producing an image that is relayed back to the camera 450. The camera 450 of the laparoscope 105 allows the user (e.g., a doctor, nurse, medical practitioner, a medical professional, etc.) to view inside the patient's body using a monitor.

Further, the medical professional views the camera 450, which displays the relayed image, and observes debris or an obstruction (including body fluids etc.). The user activates the power switch 120 (e.g., a toggle switch, a press button switch). When the power switch 120 is activated, the power source 110 provides electrical power to other components of the system for ultrasonically cleaning a laparoscopic lens 400, specifically the piezo driver 134. Upon receipt of electrical power, the piezo driver 134 transmits an activation signal to the piezoelectric transducer 170. The voltage from the activation signal is applied to the piezoelectric material 174 of the piezoelectric transducer 170, which begins to expand and compress at an ultrasonic frequency equivalent to the frequency of the activation signal waveform. The piezoelectric material produces acoustic waves, in the form of pulses, which are projected forward. The acoustic waves strike the debris or obstruction located on the optic window 180. The ultrasonic vibrations of the piezoelectric transducer 170 displaces the debris or obstructions obscuring the hydrophobic-coated optic window 180. When the user switches the power switch 120 to an “off” position, the power source 110 stops providing electrical power to the components for the system for ultrasonically cleaning a laparoscopic lens cap 400. As a result, the piezo driver 134 does not transmit an activation signal and the piezoelectric transducer 170 does not emit vibrations at an ultrasonic frequency.

In one or more embodiments, the system for ultrasonically cleaning a laparoscopic lens 400 is configured to fit laparoscopes of all angles (including 0°, 30°, 45° degrees) and all diameters.

In one or more embodiments, the system for ultrasonically cleaning a laparoscopic lens cap 400 is frictionally affixed to other surgical instruments with lens including, but not limited to, endoscopes, arthroscopes, resectoscopes, ureteroscopes, bronchoscopes, cystoscopes, nephroscopes, sinus scopes, etc.

In an alternative embodiment, the hydrophobic-coated optic window 180 is coated in other hydrophobic or oleophobic coatings (e.g., Dexide™ FRED™ Anti-Fog Solution).

In an alternative embodiment, the laparoscopic lens cap 410 is pressed onto the laparoscope 150, adhesively bonded or otherwise affixed using another method of mechanical positive retention.

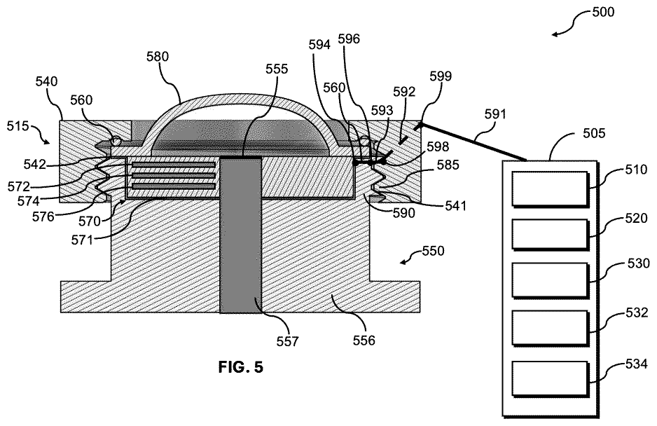

FIG. 5 illustrates a cross-section view of a laparoscopic lens cap with an ultrasonic cleaning system 500 according to an embodiment of the present invention. The laparoscopic lens cap with an ultrasonic cleaning system 500 includes a power system 505, a laparoscopic lens cap 515, and a modified laparoscope 550. The laparoscopic lens cap with an ultrasonic cleaning system 500 also includes an interior transducer wire 593, a housing wire 592, and an exterior wire 591. The laparoscopic lens cap 515 includes an exterior housing 540, a hydrophobic-coated optic window 580, a piezoelectric transducer 570, an optic window recess 542, a lens cap thread-engaged component 590, a cap-sealing O-ring 560, an interior transducer housing wire 594, the laparoscope wire aperture 596, lens cap wire aperture 598, an exterior wire aperture 599 and an interior housing wall 541. The piezoelectric transducer 570 includes a lower electrode 572, a piezoelectric material 574, an upper electrode 576, and a cylindrical transducer housing 571. The modified laparoscope 550 includes a laparoscopic outer wall 556, a laparoscopic interior 557, a laparoscope thread-engaged component 585, and an objective lens 555. The power system 505 includes a power source 510, a power switch 520, a transducer control system 530, a printed circuit board (PCB) 532, and a piezo driver 534.

In the laparoscopic lens cap with an ultrasonic cleaning system cap 500, the power source 510, the power switch 520, the transducer control system 530, the PCB 532, the piezo driver 534, and the piezoelectric transducer 570 are in a wired and electrical connection with each other. The cylindrical transducer housing 571 further comprises an interior transducer wire aperture 594. The piezoelectric transducer 570 is mechanically connected to the interior transducer wire 593 through the interior transducer wire aperture 594, which passes through the cylindrical transducer housing 571. The interior transducer wire 593 is connected to a housing wire 592 that is positioned within the exterior housing 540. Specifically, the interior transducer wire 593 connects to the housing wire 592 through the laparoscopic wire aperture 596 that is positioned within the laparoscopic thread-engaged component 585. When the laparoscopic thread-engaged component 585 is in threaded engagement with the lens cap thread-engaged component 590, the housing wire 592 is mechanically connected to the exterior wire 591, which passes through the lens cap thread-engaged component via the lens cap wire aperture 598. The exterior wire 591 passes through the exterior wire aperture 599 and is mechanically connected to the power system 505.

The piezoelectric transducer 570 includes a lower electrode 572, a piezoelectric material 574, and an upper electrode 576; the lower electrode 572, the piezoelectric material 574, and the upper electrode 576 are positioned inside the cylindrical transducer housing 571. The cylindrical transducer housing 571 is mechanically affixed to the interior housing wall 541.

The cylindrical transducer housing 571 is also mechanically affixed to the laparoscopic outer wall 556 when the laparoscope thread-engaged component 585 is in threaded engagement with the lens cap thread-engaged component 590, forming a watertight seal between the cylindrical transducer housing 571 and the laparoscopic outer wall 556. The exterior housing 540 is in mechanical connection with the hydrophobic-coated optic window 580. The hydrophobic-coated optic window 580 is held in place by the exterior housing 540 and the interior housing wall 541 such that the hydrophobic-coated optic window 580 is positioned in the optic window recess 542. The cap-sealing O-ring 560 holds the hydrophobic-coated optic window 580 in place, further affixing the hydrophobic-coated optic window 580 in the optic window recess 542. The exterior housing 540 is also in mechanical connection with the modified laparoscope 550, specifically at the laparoscopic outer wall 556. The laparoscopic outer wall 556 is mechanically connected to laparoscopic interior 557. The laparoscopic interior 557 is mechanically coupled with the objective lens 555.

In operation, the modified laparoscope 550 is modified to include male threads (e.g., the laparoscope thread-engaged component 585) that allow the lens cap thread-engaged component 590 to be in threaded engagement with the laparoscope thread-engaged component 585. When the exterior housing 540 is threaded onto the modified laparoscope 550, the hydrophobic-coated optic window 580 is radially offset from the objective lens 555 of the modified laparoscope 550 such that the hydrophobic-coated optical window 580 covers the objective lens 555 of the modified laparoscope 550. Next, the modified laparoscope 550 is inserted into a patient's body by a user such as a medical professional. When the hydrophobic-coated optic window 580 is obscured by an obstruction or debris (e.g., blood, tissue, or other bodily fluids) on the surface of the hydrophobic-coated optical window 580, the user activates the power switch 520 into an “on” position. Once the power switch is activated, the power source 510 begins providing electrical power to the PCB 532 and the transducer control system 530. In turn, the transducer control system 530 provides electrical power to the piezo driver 534. The piezo driver 534 further provides electrical power to the piezoelectric transducer 570.

In further operation, the piezoelectric transducer 570 includes a lower electrode 576 and an upper electrode 572 with a piezoelectric material 574 (e.g., barium titanate, lead zirconate titanate (PZT), lead titanate, Rochelle salt) situated between the two electrodes. Once powered, the piezoelectric material resonates at an ultrasonic frequency equivalent to the frequency of the voltage signal sent from the piezo driver, with a voltage high enough to cause sufficient mechanical displacement (i.e. generally 40-60V), causing the piezoelectric transducer to vibrate at an ultrasonic frequency (e.g., 20 kHz-400 kHz). As the piezoelectric transducer 570 ultrasonically vibrates, the debris/obstruction on the hydrophobic-coated optic window 580 is displaced from the surface of the hydrophobic-coated optic window 580. Alternatively, the debris/obstruction no longer adheres to the lens/is physically or mechanically separated from the lens. As the debris/obstruction is displaced, the funnel guard 595 guides the debris away from the hydrophobic-coated optic window 180 and back into the body.

In the preferred embodiment, the modified laparoscope 550 is preferably modified using a tapping process, including a computer numerical control (CNC) lathe, that involves clamping a laparoscope on a chuck and threading the stem/cannula of the laparoscope, producing the laparoscope thread-engaged component. The laparoscope thread-engaged component 585 is in threaded engagement with the lens cap thread-engaged component 590 such that the hydrophobic-coated optic window 580 is radially offset from the objective lens 555.

In view of the foregoing teaching, one or more embodiments of the present invention provide numerous advantages over other known systems, methods, and devices for cleaning a laparoscopic lens. Importantly, the system for ultrasonically cleaning a laparoscopic lens cap 100 minimizes cleaning time, saving a significant amount of time for the surgeons.

The system for ultrasonically cleaning a laparoscopic lens cap 100 minimizes cleaning time. Unlike other prior art systems (described above), the user does not have to remove the laparoscope from the patient's body in order to clean the laparoscopic lens using the system for ultrasonically cleaning a laparoscopic lens cap 100. Similarly, other prior art systems do not use a sheath or lens cover that includes a transducer. Because the system for ultrasonically cleaning a laparoscopic lens 100 uses a transducer, any debris or obstruction on the laparoscopic lens is removed within five seconds. Other prior art systems also do not defog the laparoscopic lens using a hydrophobic and/or oleophobic coating such as PTFE coating which is resistant to fogging.

Additionally, the system for ultrasonically cleaning a laparoscopic lens 100 reduces wear of the surgical scope compared to other prior art systems that require removal of the scope from the patient's body before cleaning. Unlike the system for Baril et al., the system for ultrasonically cleaning a laparoscopic lens 100 does require a wiper blade attachment. As a result, the risk of an attachment breaking off and harming the patient's body will not occur. Similarly, the system for ultrasonically cleaning a laparoscopic lens 100 does not include the use of microfilm within the patient's body, which also risks contaminating the patient's body.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. An ultrasonic cleaning system for laparoscopic instruments, said system comprising:
a laparoscope comprising a lens positioned in a laparoscope housing; and
a removable housing comprising:
an optic window, wherein said removable housing is removably affixed to said laparoscope housing so that said lens includes a field of view through said optic window; and
an ultrasonic transducer system including an ultrasonic transducer, wherein said ultrasonic transducer is vibrationally coupled to said optic window, wherein vibration produced by said ultrasonic transducer causes material positioned in contact with said optic window to be displaced from said optic window.

2. The ultrasonic cleaning system for laparoscopic instruments of claim 1, wherein said removable housing is in the threaded engagement, or mechanically retained positively using another method, with said laparoscopic housing so that said removable housing and said laparoscopic housing is removably affixed.

3. The ultrasonic cleaning system for laparoscopic instruments of claim 1, wherein said ultrasonic transducer system further comprises a transducer control system, wherein said transducer control system receives electrical power from a piezoelectric driver which receives power from a power supply and further transmits said electrical power to said ultrasonic transducer.

4. The ultrasonic cleaning system for laparoscopic instruments of claim 3, wherein said ultrasonic transducer ultrasonically vibrates at a frequency equivalent to said voltage signal of said piezoelectric driver.

5. The ultrasonic cleaning system for laparoscopic instruments of claim 1, wherein said optic window includes a hydrophobic and oleophobic coating or hydrophobic coating, wherein said coating may be a PTFE coating.

6. The ultrasonic cleaning system for laparoscopic instruments of claim 1, wherein said removable housing is mechanically affixed to a funnel guard, wherein said funnel guard guides said material displaced by said ultrasonic vibrations.

7. The ultrasonic cleaning system for laparoscopic instruments of claim 1, wherein said ultrasonic cleaning system further includes a power switch, wherein said power switch activates said vibrations of said ultrasonic transducer.

8. The ultrasonic cleaning system for laparoscopic instruments of claim 1, wherein said ultrasonic transducer system further comprises a piezoelectric sensor, a piezoelectric actuator, an integrated circuit, a PCB, and a piezo driver, wherein said piezoelectric sensor experiences pressure change from said material positioned in contact with said optic window, wherein said piezoelectric sensor transmits an electrical signal to said integrated circuit via said PCB, which then sends a signal to said piezo driver, wherein said piezo driver further transmits an activation signal to said piezoelectric actuator, wherein said piezoelectric actuator activates and ultrasonically vibrates in response.

9. A method for ultrasonically cleaning an endoscopic device, said method including:
affixing a removable housing having an optic window to an endoscope housing having a lens, wherein said optic window is positioned so that said lens includes a field of view through said optic window; and
activating an ultrasonic transducer system in said removable housing to produce an ultrasonic vibration, wherein said ultrasonic transducer system is vibrationally coupled to said optic window so as to induce vibration of said optic window to displace material positioned in contact with said optic window.

10. The method of claim 9, further including controlling said ultrasonic transducer using an electrical power switch.

11. The method of claim 9, further including transmitting electrical power from a battery to said ultrasonic transducer, wherein said ultrasonic transducer produces ultrasonic vibrations, wherein said material positioned in contact with said optic window is displaced.

12. The method of claim 9, further including detecting said material positioned in contact with said optic window using a piezoelectric sensor connected to said power supply, in unidirectional communication with a driver,
wherein generating an activation signal is performed by said driver, in unidirectional communication with said ultrasonic transducer,
wherein transmitting said activation signal to said ultrasonic transducer is performed by said driver, and
wherein generating ultrasonic vibrations is performed by said ultrasonic transducer when said activation signal is received.

13. The method of claim 9, further including a funnel guard positioned to further displace said material displaced by said ultrasonic vibrations away from said optic window.

14. A system for cleaning a laparoscopic lens comprising:
a laparoscope including a laparoscopic lens cap; wherein said laparoscopic lens cap comprises an optic window, wherein said optic window is coated in a hydrophobic coating, wherein said hydrophobic coating resists water condensation.

15. The system of claim 14, wherein said hydrophobic coating may be a PTFE coating.

16. The system of claim 14, wherein said lens cap further includes a lens cap thread-engaged component and said laparoscope includes a laparoscope thread-engaged component, wherein said lens cap thread-engaged component is in threaded engagement with said laparoscope thread-engaged component, wherein said lens cap is removably affixed to said laparoscope.

17. The system of claim 14, wherein said lens cover is connected mechanically to said laparoscope using adhesive bonding.

18. The system of claim 14, further including an ultrasonic transducer and power supply from wall power adapter, wherein said power supply transmits electrical power to said ultrasonic transducer, wherein said ultrasonic transducer receives said electrical power and produces vibrations, wherein said ultrasonic vibrations cause material positioned in contact with said lens cap to be displaced from said optic window.

19. The system of claim 18 further including a transducer control system and driver, wherein said transducer control system is mechanically connected to said lens cap, wherein said power supply transmits electrical power to said transducer control system, wherein said transducer control system transmits said electrical power to said driver, wherein said driver further transmits electrical power to said ultrasonic transducer, wherein said ultrasonic transducer receives said electrical power and produces vibrations, wherein said vibrations remove material positioned in contact with said optic window.
